(19) United States
(12) Patent Application Publication
(10) Pub. No.: US 2026/0098938 A1
(43) Pub. Date: Apr. 9, 2026

(54) ELECTRONIC DEVICE AND METHOD OF PROCESSING MOTION SIGNAL

(71) Applicant: Wistron Corporation, New Taipei City (TW)

(72) Inventors: Kaijen Cheng, New Taipei City (TW); Yao Tsung Chang, New Taipei City (TW); Yin Yu Chen, New Taipei City (TW)

(73) Assignee: Wistron Corporation, New Taipei City (TW)

(21) Appl. No.: 18/961,381

(22) Filed: Nov. 26, 2024

(30) Foreign Application Priority Data
Oct. 9, 2024 (TW) 113138509

(51) Int. Cl. G01S 7/41; A61B 5/11

(52) U.S. Cl. CPC G01S 7/415; A61B 5/1116

(57) ABSTRACT

An electronic device and a method of processing a motion signal are provided. The method includes: performing detection through a radar to obtain a dynamic signal; executing continuous wavelet transform on the dynamic signal to obtain a scalogram; dividing the scalogram to generate multiple samples; clustering the samples into a first cluster and a second cluster; sampling the motion signal from the dynamic signal according to the first cluster; and outputting the motion signal.

### CROSS-REFERENCE TO RELATED APPLICATION

This application claims the priority benefit of Taiwan application serial no. 113138509, filed on Oct. 9, 2024. The entirety of the above-mentioned patent application is hereby incorporated by reference herein and made a part of this specification.

### BACKGROUND

### Technical Field

The disclosure relates to a signal processing technology, and in particular to an electronic device and a method of processing a motion signal.

### Description of Related Art

Currently, medical personnel may use a sensor to detect motion behavior of a subject within a time period, and determine the health condition of the subject according to the detection result. For example, medical personnel may determine whether the motion ability of the subject is poor according to the detection result, thereby determining whether the subject suffers from sarcopenia. However, traditional methods can only evaluate the overall motion behavior of the subject within a time period, but cannot evaluate individual actions of the subject. Therefore, detection results of motion behavior generated by the traditional methods are not very accurate and reliable.

### SUMMARY

The disclosure provides an electronic device and a method of processing a motion signal, which can extract the motion signal related to an action of a subject from a radar signal.

A method of processing a motion signal according to an embodiment of the disclosure includes the following steps. Detection is performed through a radar to obtain a dynamic signal. Continuous wavelet transform is executed on the dynamic signal to obtain a scalogram. The scalogram is divided to generate multiple samples. The samples are clustered into a first cluster and a second cluster. The motion signal is sampled from the dynamic signal according to the first cluster. The motion signal is output.

An electronic device of processing a motion signal according to an embodiment of the disclosure includes a transceiver and a processor. The transceiver is communicatively connected to a radar. The processor is coupled to the transceiver and is configured to execute the following. Detection is performed through a radar to obtain a dynamic signal. Continuous wavelet transform is executed on the dynamic signal to obtain a scalogram. The scalogram is divided to generate multiple samples. The samples are clustered into a first cluster and a second cluster. The motion signal is sampled from the dynamic signal according to the first cluster. The motion signal is output through the transceiver.

Based on the above, the electronic device of the disclosure may execute abnormality detection and noise filtering on the radar signal, and may generate an analysis result according to the processed radar signal. The analysis result generated according to the method of the disclosure may accurately indicate the sample of the radar signal corresponding to each action of the subject. A user may evaluate individual actions of the subject based on the analysis result.

### BRIEF DESCRIPTION OF THE DRAWINGS

FIG. 1 is a schematic diagram of an electronic device of processing a motion signal according to an embodiment of the disclosure.

FIG. 2 is a schematic diagram of using a radar to detect motion behavior of a subject according to an embodiment of the disclosure.

FIG. 3 is a schematic diagram of a mapping relationship between an action of a subject and a radar signal according to an embodiment of the disclosure.

FIG. 4 is a flowchart of sampling a motion signal according to an embodiment of the disclosure.

FIG. 5 is a schematic diagram of a reconstructed dynamic signal according to an embodiment of the disclosure.

FIG. 6 is a schematic diagram of converting a dynamic signal into a scalogram according to an embodiment of the disclosure.

FIG. 7 is a flowchart of generating an analysis result according to an embodiment of the disclosure.

FIG. 8 is a schematic diagram of a motion signal and a slope signal according to an embodiment of the disclosure.

FIG. 9 is a flowchart of updating an analysis result according to an embodiment of the disclosure.

FIG. 10 is a schematic diagram of a motion signal, a slope signal, a distance signal, and a reference signal according to an embodiment of the disclosure.

FIG. 11 is a schematic diagram of a result of dynamic time warping according to an embodiment of the disclosure.

FIG. 12 is a flowchart of a method of processing a motion signal according to an embodiment of the disclosure.

### DESCRIPTION OF THE EMBODIMENTS

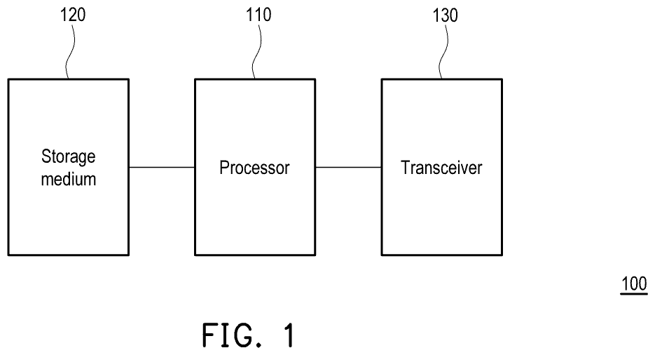

FIG. 1 is a schematic diagram of an electronic device 100 of processing a motion signal according to an embodiment of the disclosure. The electronic device 100 may include a processor 110, a storage medium 120, and a transceiver 130.

The processor 110 is, for example, a central processing unit (CPU), other programmable general-purpose or specific-purpose micro control units (MCU), microprocessors, digital signal processors (DSP), programmable controllers, application specific integrated circuits (ASIC), graphics processing units (GPU), image signal processors (ISP), image processing units (IPU), arithmetic logic units (ALU), complex programmable logic devices (CPLD), field programmable gate arrays (FPGA), other similar elements, or a combination of the above elements. The processor 110 may be coupled to the storage medium 120 and the transceiver 130, and access and execute multiple modules and various applications stored in the storage medium 120.

The storage medium 120 is, for example, any type of fixed or removable random access memory (RAM), read-only memory (ROM), flash memory, hard disk drive (HDD), solid state drive (SSD), similar elements, or a combination of the above elements to store the modules or the various applications that may be executed by the processor 110.

The transceiver 130 transmits or receives signals wirelessly or wired. The transceiver 130 may also execute low noise amplification, impedance matching, frequency mixing, up or down frequency conversion, filtering, amplification, and similar operations. The processor 110 may be communicatively connected to a radar (not shown) through the transceiver 130. The radar may include, for example, a millimeter wave (mmWave) radar or a frequency modulated continuous wave (FMCW) radar. The processor 110 may receive a detection result of the radar from the radar.

The electronic device 100 may detect a specific action executed by a subject through the radar, and generate an analysis result. The analysis result may include a mapping relationship between the action executed by the subject and a sample of a radar signal. For example, in order to determine whether the subject suffers from sarcopenia, a user of the electronic device 100 may instruct the subject to stand up and sit down repeatedly, and use the radar to detect motion behavior of the subject to generate the analysis result. The analysis result may include a mapping relationship between a standing up action of the subject and the sample of the radar signal, and may include a mapping relationship between a sitting action of the subject and the sample of the radar signal.

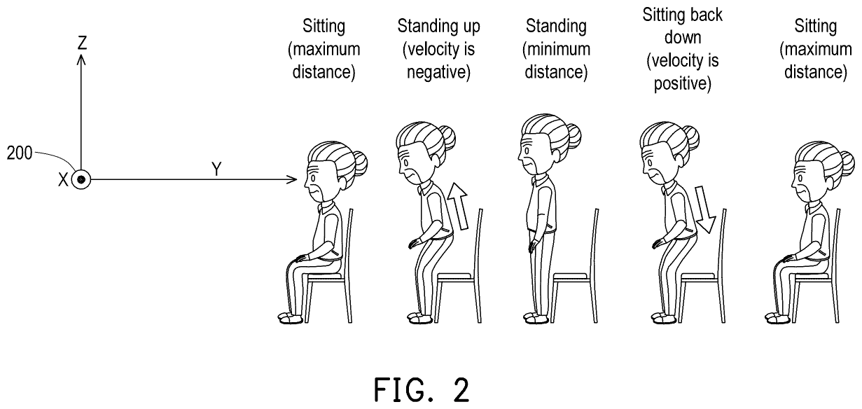

FIG. 2 is a schematic diagram of using a radar 200 to detect motion behavior of a subject according to an embodiment of the disclosure. The radar 200 may be configured to detect the motion behavior of the subject, thereby generating a dynamic signal or a distance signal. The dynamic signal, for example, includes a velocity signal, an acceleration signal, or a Doppler signal. The definition of the Doppler signal is as shown in equation (1), where x represents the distance in the X direction, \dot{x} represents the velocity in the X direction, y represents the distance in the Y direction, \dot{y} represents the velocity in the Y direction, z represents the distance in the Z direction, and ż represents the velocity in the Z direction.

$$\frac{x \dot{x} + y \dot{y} + z \dot{z}}{\sqrt{{x}^{2} + {y}^{2} + {z}^{2}}} \qquad ( 1 )$$

In an embodiment, the radar 200 is located at the coordinate origin, and the boresight of the radar 200 is the Y-axis. The subject sequentially executes five actions: sitting, standing up, standing, sitting back down, and sitting. When the subject is sitting, the distance signal detected by the radar 200 may represent that a distance between the subject and the radar 200 (that is, the distance on the Y-axis) is maximum. When the subject stands up, the velocity signal detected by the radar 200 may represent that the velocity of the subject is negative (that is, the subject moves toward the coordinate origin along the Y-axis direction). When the subject is standing, the distance signal detected by the radar 200 may represent that the distance between the subject and the radar 200 is minimum. When the subject sits back down on a chair, the velocity signal detected by the radar 200 may represent that the velocity of the subject is positive. When the subject is sitting, the distance signal detected by the radar 200 may represent that the distance between the subject and the radar 200 is maximum. The electronic device 100 may generate the analysis result according to the detection result of the radar signal. The analysis result may indicate the sample of the radar signal corresponding to the standing up action or sitting back down action of the subject.

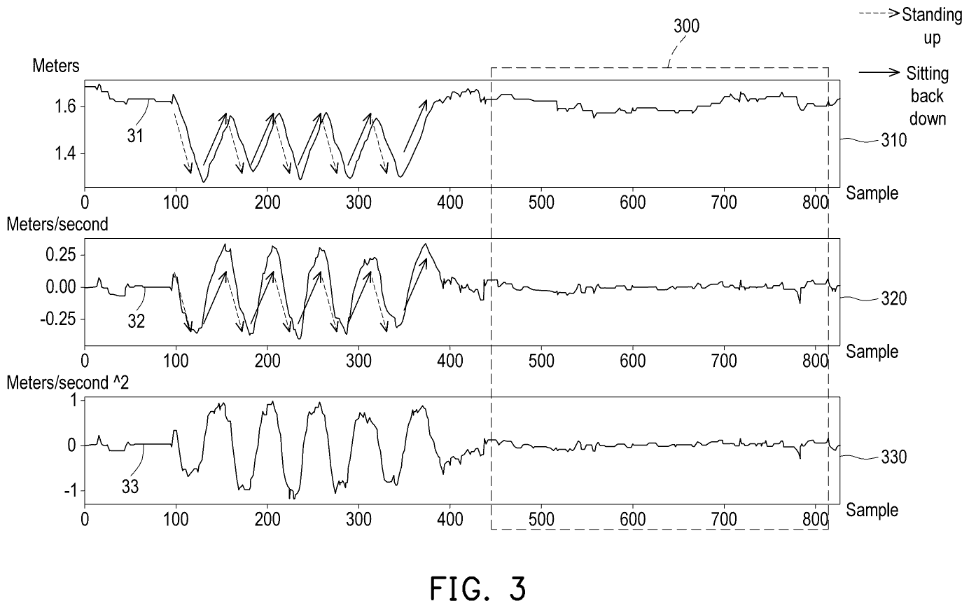

FIG. 3 is a schematic diagram of a mapping relationship between an action of a subject and a radar signal according to an embodiment of the disclosure. The radar signal may include a distance signal 310, a velocity signal 320, or an acceleration signal 330 generated by the radar 200. The analysis result generated by the electronic device 200 according to the radar signal may indicate a sample of the distance signal 310 or a sample of the velocity signal 320 corresponding to the standing up action or the sitting back down action of the subject, as shown in FIG. 3. After five cycles of standing up and sitting back down, during a period of a time window 300, the subject remains seated. Therefore, the analysis result may indicate that multiple samples in the time window 300 do not correspond to any action.

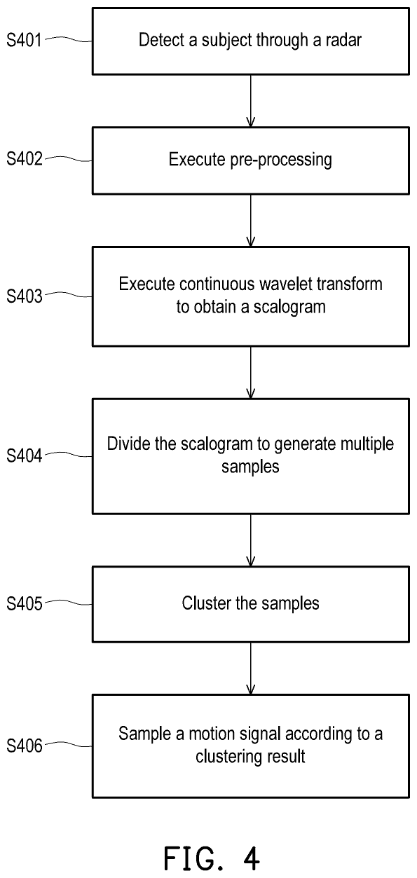

FIG. 4 is a flowchart of sampling a motion signal according to an embodiment of the disclosure. The process may be implemented by the electronic device 100 shown in FIG. 1. In step S401, the processor 110 may detect the subject through the radar 200 to obtain the dynamic signal. The dynamic signal may include the velocity signal, the acceleration signal, or the Doppler signal. In an embodiment, a signal detected by the radar 200 may further include the distance signal.

In an embodiment, the radar 200 may be configured such that the value of the dynamic signal (or the distance signal) increases during a period of the subject executing a specific action, and such that the value of the dynamic signal (or the distance signal) decreases during a period of the subject executing another action. For example, the radar 200 may be configured such that the value of the velocity signal (or the distance signal) decreases when the subject stands up, and such that the value of the velocity signal (or the distance signal) increases when the subject sits back down, as shown in FIG. 3.

In step S402, the processor 110 may execute pre-processing on the dynamic signal to filter noise in the dynamic signal or extract important features of the dynamic signal.

In an embodiment, the processor 110 may execute singular spectrum analysis (SSA) on the dynamic signal to reconstruct the dynamic signal. First, the processor 110 may convert a time series of the dynamic signal into a matrix (for example, a Hankel matrix), and then decompose the matrix through singular value decomposition (SVD), thereby obtaining a singular value representing a principal component of the series and a corresponding singular vector. The principal component may include characteristics such as trends, periodicities, or random fluctuations of the dynamic signal. The processor 110 may select one or more singular vectors to decompose the time series into a sum of individual components to obtain a reconstructed dynamic signal.

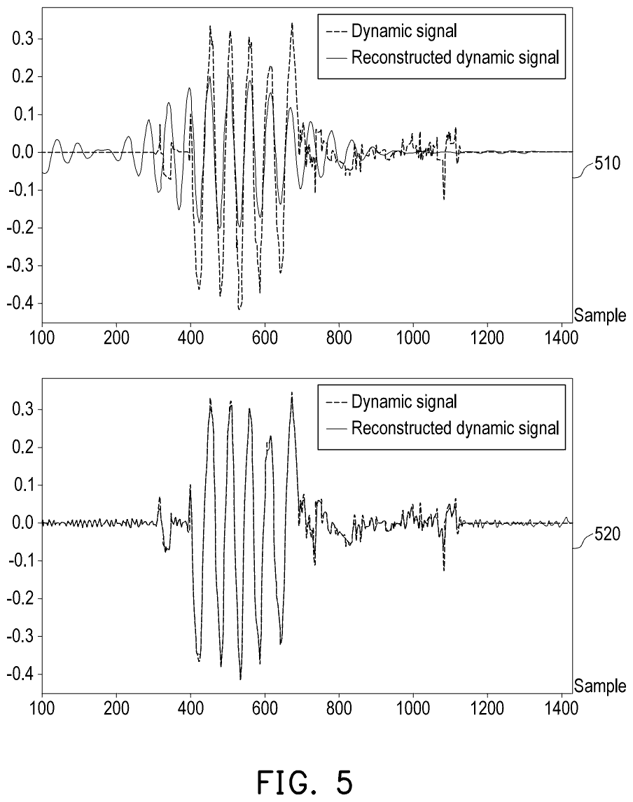

When reconstructing the dynamic signal, if the processor 110 selects more singular vectors, the reconstructed dynamic signal will be closer to original data. FIG. 5 is a schematic diagram of a reconstructed dynamic signal according to an embodiment of the disclosure. A simulation diagram 510 is a dynamic signal reconstructed using 3 singular vectors, and a simulation diagram 520 is a dynamic signal reconstructed using 100 singular vectors. In the case where too few singular vectors are used, the reconstructed dynamic signal may only include information of more important features and lack details in the original data. When reconstructing the dynamic signal, the user needs to weigh the level of importance of the accuracy and the velocity of data processing to determine the number of singular vectors to be used.

Returning to FIG. 4, in step S403, the processor 110 may execute continuous wavelet transform (CWT) on the dynamic signal to obtain a scalogram or a wavelet coefficient power diagram. In an embodiment, the processor 110 may execute the continuous wavelet transform on the dynamic signal using a wavelet function including a cgau6 function.

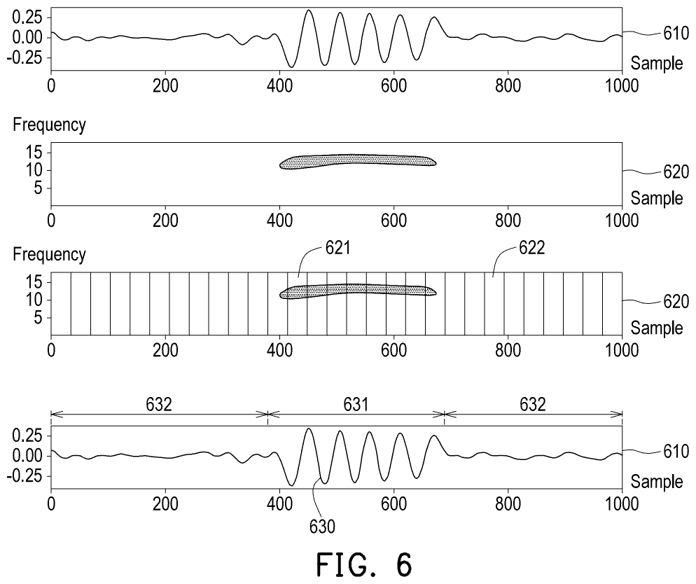

FIG. 6 is a schematic diagram of converting a dynamic signal into a scalogram according to an embodiment of the disclosure. The processor 110 may execute the continuous wavelet transform on a dynamic signal 610 to generate a scalogram 620. The abscissa axis of the scalogram 620 may be time (or sample), the ordinate axis may be frequency, and the scalogram 620 may use shades of color to represent an intensity of a sample at a specific frequency. Taking the scalogram 620 as an example, an area corresponding to samples 400 to samples 600 and frequency 10 to frequency 15 in the scalogram 620 has a darker color. Therefore, the processor 110 may determine that the dynamic signal has greater intensity in the area.

Returning to FIG. 4, in step S404, the processor 110 may divide the scalogram in the time domain to generate multiple samples. Taking the scalogram 620 as an example, the processor 110 may divide the scalogram 620 in the time domain to generate multiple samples including a sample 621 and a sample 622.

In step S405, the processor 110 may cluster the samples to obtain two clusters: a first cluster and a second cluster. In an embodiment, the processor 110 may cluster the samples according to k-means clustering.

The first cluster may include one or more samples that represent more active motions of the subject (for example, samples when the subject executes actions such as standing up or sitting back down repeatedly), and the second cluster may include one or more samples that represent less active motions of the subject (for example, a sample when the subject is sitting on the chair and resting). Taking the scalogram 620 as an example, the processor 110 may assign the sample 621 representing the more active motion of the subject to the first cluster, and assign the sample 622 representing the less active motion of the subject to the second cluster. The number of samples in the first cluster may be less than the number of samples in the second cluster. Therefore, after executing clustering, the processor 110 may determine that a cluster including fewer samples is the first cluster, and a cluster including more samples is the second cluster.

In step S406, the processor 110 may sample the motion signal from the dynamic signal according to the first cluster, and the motion signal represents a signal during a time period of more active motion behavior of the subject. The processor 110 may output the motion signal through the transceiver 130 for user reference.

Taking FIG. 6 as an example, the processor 110 may obtain one or more continuous time periods corresponding to one or more samples in the first cluster, and set the one or more continuous time periods as a time window 631. The processor 110 may sample a motion signal 630 from the dynamic signal 610 according to the time window 631. On the other hand, the processor 110 may obtain one or more time periods corresponding to one or more samples in the second cluster, and set the one or more time periods as a time window 632. The time window 632 represents a time period of less active motion behavior of the subject.

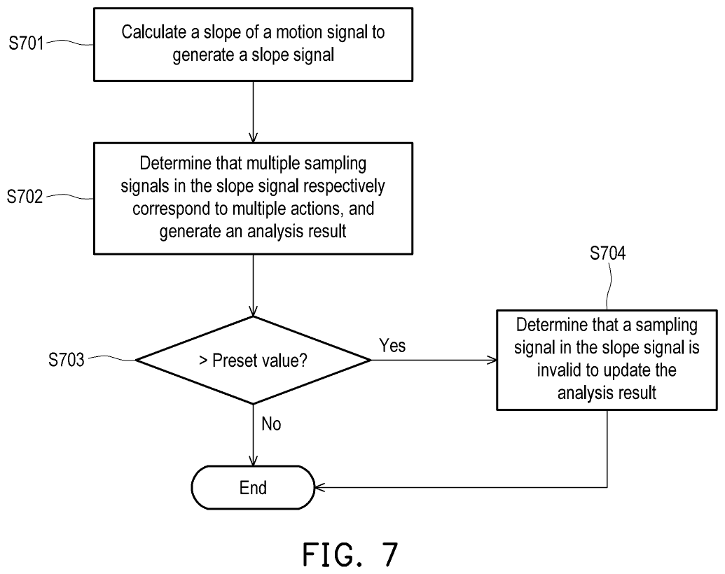

After obtaining the motion signal representing the overall motion behavior of the subject during a specific time period, the processor 110 may further match each action of the subject with each sampling signal in the motion signal, thereby generating the analysis result. The analysis result may indicate the action of the subject corresponding to each sampling signal in the motion signal. FIG. 7 is a flowchart of generating an analysis result according to an embodiment of the disclosure. The process may be implemented by the electronic device 100 shown in FIG. 1.

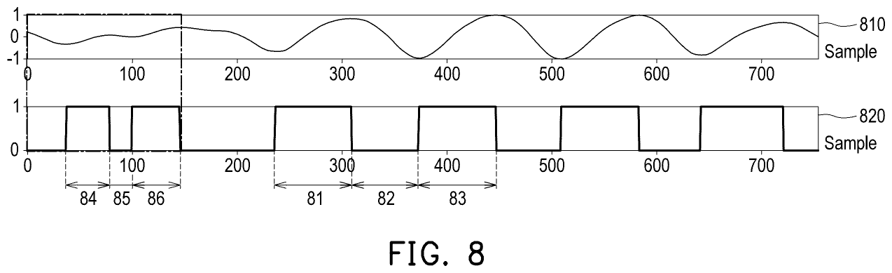

In step S701, the processor 110 may calculate the slope of the motion signal to generate a slope signal. As shown in FIG. 8, the processor 110 may calculate a slope of a motion signal 810 to generate a slope signal 820. In an embodiment, the processor 110 may normalize the slope signal 820, so that slope signal 820 has a value between 0 and 1.

In step S702, the processor 110 may determine that multiple sampling signals in the slope signal respectively correspond to multiple actions of the subject, and generate the analysis result. The processor 110 may output the analysis result through the transceiver 130 for user reference.

In an embodiment, the processor 110 may classify the sampling signals into a first classification corresponding to a first action (for example, the sitting back down action) and a second classification corresponding to a second action (for example, the standing up action). If each sampling point of the sampling signal is greater than a threshold, the processor 110 may determine that the sampling signal corresponds to the first classification. If each sampling point of the sampling signal is less than or equal to a sampling threshold, the processor 110 may determine that the sampling signal corresponds to the second classification.

Taking the slope signal 820 as an example, it is assumed that the motion signal is the Doppler signal. Based on the rule that “the distance and the velocity decrease when the subject stands up, and the distance and the velocity increase when the subject sits back down”, the processor 110 may determine that four sampling signals respectively corresponding to a time period 81, a time period 83, a time period 84, and a time period 86 correspond to the sitting back down action in response to the four sampling signals being greater than the sampling threshold. On the other hand, the processor 110 may determine that two sampling signals respectively corresponding to a time period 82 and a time period 85 correspond to the standing up action in response to the two sampling signals being less than or equal to the sampling threshold. The processor 110 may determine the action corresponding to each sampling signal in the slope signal, and generate the analysis result.

In an embodiment, the processor 110 may determine whether the sampling signal is valid according to the time period of the sampling signal. If the time period of the sampling signal is greater than a time threshold T1, the processor 110 may determine that the sampling signal is valid to generate the analysis result. If the time period of the sampling signal is less than or equal to the time threshold T1, the processor 110 may determine that the sampling signal is invalid. Generally speaking, the time it takes for the subject to execute the standing up and sitting back down action once is about 2 seconds. In other words, the standing up action and the sitting back down action respectively take about 1 second. Accordingly, the processor 110 may, for example, set the time threshold T1 to 0.066 seconds. If the time period of a segment of the sampling signal corresponding to the sitting back down action is greater than 0.066 seconds, the processor 110 may determine that the sampling signal is valid. Taking the slope signal 820 as an example, the processor 110 may determine that the sampling signal representing the sitting back down action corresponding to the time period 81 is valid based on the time period 81 being greater than the time threshold T1.

In an embodiment, if there is a short sampling signal corresponding to another action between two sampling signals corresponding to the same action, the sampling signal may be caused by noise. The processor 110 may combine the sampling signal with the two sampling signals into a single sampling signal. Specifically, it is assumed that the slope signal includes a first sampling signal, a second sampling signal, and a third sampling signal. The first sampling signal and the third sampling signal correspond to the classification of the first action, and the second sampling signal between the first sampling signal and the third sampling signal corresponds to the classification of the second action. In response to the time period of the second sampling signal being less than or equal to a time threshold T2, the processor 110 may update the slope signal to combine the first sampling signal, the second sampling signal, and the third sampling signal into the single sampling signal to match the first action. The time threshold T2 may be 0.5 times the time threshold T1.

Taking the slope signal 820 as an example, the processor 110 may determine that the sampling signals corresponding to the time period 84 and the time period 86 represent the sitting back down action, and the sampling signal corresponding to the time period 85 represents the standing up action. If the time period 85 is less than or equal to the time threshold T2, the processor 110 may determine that the sampling signal of the time period 85 is affected by noise. The processor 110 may determine that the time period 84, the time period 85, and the time period 86 correspond to a single sitting back down action, and may update the analysis result based on the determination result.

In an embodiment, if the slope signal is the distance signal, the velocity signal, or the Doppler signal, the processor 110 may determine whether the sampling signal in the slope signal is valid according to the acceleration signal. Taking the sampling signal corresponding to the time period 81 in the slope signal 820 as an example, the processor 110 may determine that the sampling signal corresponds to the sitting back down action based on the sampling signal being greater than the sampling threshold. Since the sitting back down action causes the subject to be away from the radar 200, the acceleration signal detected by the radar 200 should be positive. Accordingly, if the sampling signal of the acceleration signal within the time period 81 is positive, it represents that the sampling signal of the acceleration signal 820 in the time period 81 matches the sampling signal of the slope signal 820 in the time period 81. Accordingly, the processor 110 may determine that the sampling signal of the slope signal 820 in the time period 81 is valid. On the other hand, if the sampling signal of the acceleration signal within the time period 81 is negative, it represents that the sampling signal of the acceleration signal in the time period 81 does not match the sampling signal of the slope signal 820 in the time period 81. Accordingly, the processor 110 may determine that the sampling signal of the slope signal 820 in the time period 81 is invalid.

Returning to FIG. 7, in order to evaluate the health condition of the subject, the subject is usually asked to perform a fixed number of specific actions. If the number of sampling signals corresponding to the actions identified by the processor 110 according to the slope signal is greater than the fixed number, it represents that the slope signal may include noise. In order to improve the accuracy of the analysis result, the processor 110 may execute step S703 and step S704 to update the analysis result.

Specifically, in step S703, the processor 110 may count the number of multiple sampling signals (or multiple actions) identified by the processor 110 from the slope signal, and determine whether the number is greater than a preset value. If the number is greater than the preset value, it represents that certain sampling signals are noise. Accordingly, the processor 110 may determine in step S704 that one or more sampling signals in the slope signal are invalid to update the analysis result. If the number is less than or equal to the preset value, it represents that the sampling signals should not be noise. Accordingly, the processor 110 may not update the analysis result.

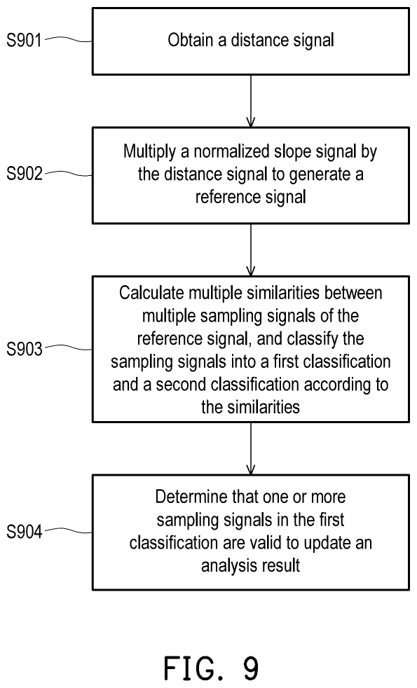

FIG. 9 is a flowchart of updating an analysis result according to an embodiment of the disclosure. The process may be implemented by the electronic device 100 shown in FIG. 1. In step S901, the processor 110 may detect the subject through the radar to obtain the distance signal. In step S902, the processor 110 may normalize the slope signal, and multiply the normalized slope signal by the distance signal to generate a reference signal.

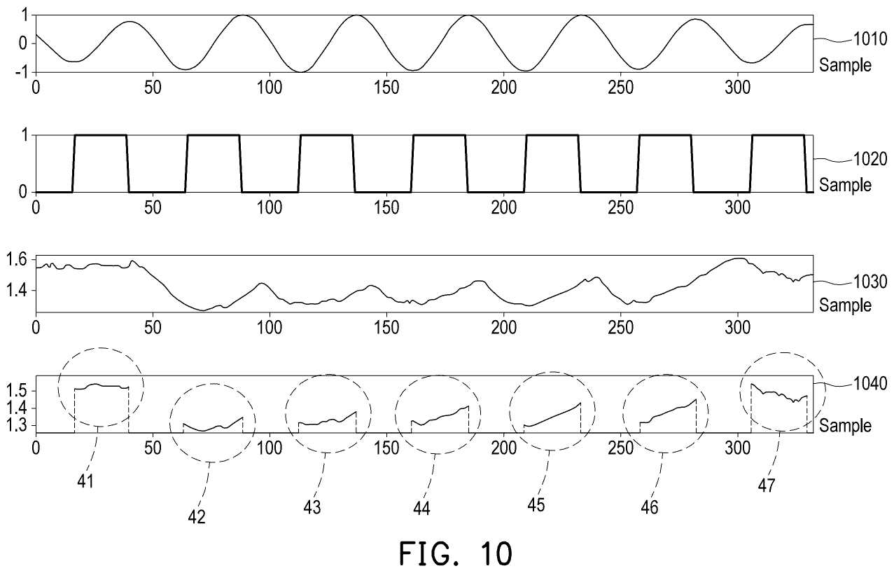

FIG. 10 is a schematic diagram of a motion signal 1010, a slope signal 1020, a distance signal 1030, and a reference signal 1040 according to an embodiment of the disclosure. After the processor 110 calculates the slope of the motion signal 1010 to obtain the slope signal 1020, and normalizes the slope signal 1020, the processor 110 may multiply the normalized slope signal 1020 by the distance signal 1030 to generate the reference signal 1040.

The processor 110 may extract one or more sampling signals from the reference signal 1040. For example, the processor 110 may extract a part of the reference signal 1040 greater than the sampling threshold as a sampling signal, such as a sampling signal 41, 42, 43, 44, 45, 46, or 47. The sampling signal extracted by the processor 110 corresponds to the action of the subject or noise.

Returning to FIG. 9, in step S903, multiple similarities between multiple sampling signals of the reference signal are calculated, and the sampling signals are classified into the first classification and the second classification according to the similarities. The sampling signals in the first classification are valid, and the sampling signals in the second classification are invalid. The valid sampling signals may be retained in the analysis result, and the invalid sampling signals may be deleted from the analysis result.

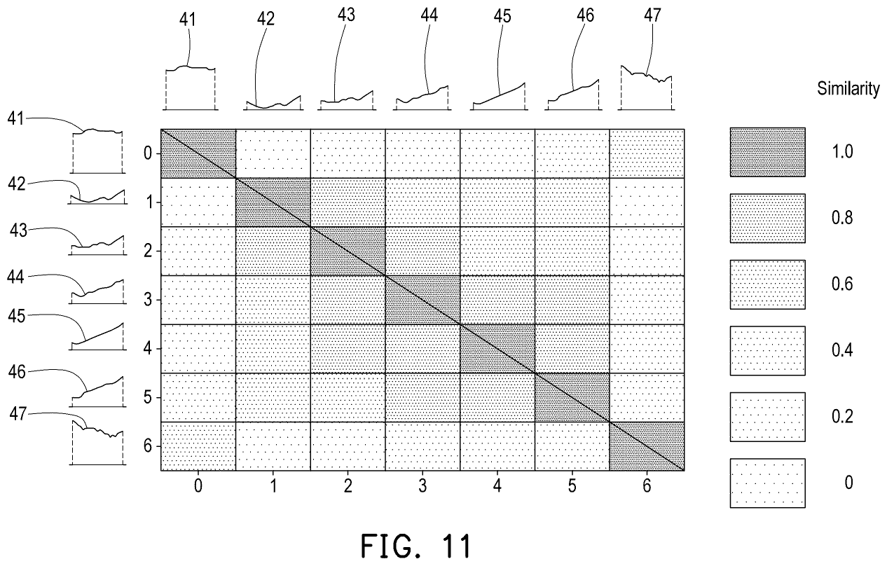

Specifically, the processor 110 may execute dynamic time warping (DTW) on the sampling signals to obtain the similarities. FIG. 11 is a schematic diagram of a result of executing dynamic time warping on the sampling signals 41, 42, 43, 44, 45, 46, and 47 according to an embodiment of the disclosure. The darker the color, the higher the similarity, and the lighter the color, the lower the similarity.

After obtaining the similarities, the processor 110 may classify the sampling signals into the first classification and the second classification according to the similarities. The processor 110 may execute agglomerative hierarchical clustering on the sampling signals according to the similarities, so that the number of samples assigned to the first classification reaches the preset value (for example, five). After the number of samples in the first classification reaches the preset value, the processor 110 may stop executing agglomerative hierarchical clustering. One or more sampling signals that have not been assigned to the first classification may be assigned to the second classification.

In step S904, the processor 110 may determine that one or more sampling signals in the first classification are valid to update the analysis result. Taking the sampling signals 41, 42, 43, 44, 45, 46, and 47 as an example, after completing agglomerative hierarchical clustering, the number of the sampling signals 42, 43, 44, 45, and 46 included in the first classification has reached the preset value, so the processor 110 may assign the sampling signals 41 and 47 that have not been assigned to the first classification to the second classification. The processor 110 may determine that the sampling signal 41 and the sampling signal 47 are invalid, and delete the sampling signal 41 and the sampling signal 47 from the analysis result. The analysis result may only retain information related to the sampling signals 42, 43, 44, 45, and 46.

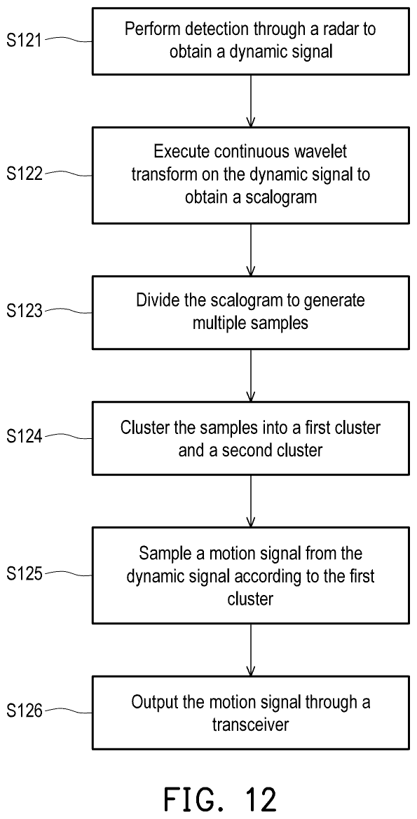

FIG. 12 is a flowchart of a method of processing a motion signal according to an embodiment of the disclosure. The method may be implemented by the electronic device 100 shown in FIG. 1. In step S121, detection is performed through a radar to obtain a dynamic signal. In step S122, continuous wavelet transform is executed on the dynamic signal to obtain a scalogram. In step S123, the scalogram is divided to generate multiple samples. In step S124, the samples are clustered into a first cluster and a second cluster. In step S125, the motion signal is sampled from the dynamic signal according to the first cluster. In step S126, the motion signal is output through a transceiver.

In summary, the electronic device of the disclosure may detect the subject in motion through the radar to obtain the dynamic signal. The electronic device may cluster the samples generated from the dynamic signal through the singular spectrum analysis, the continuous wavelet transform, etc. to determine the action of the subject corresponding to each sample. In other words, the electronic device may obtain the motion signal corresponding to each action executed by the subject, thereby generating the analysis result. The analysis result may record the mapping relationship between the motion signal and the action of the subject. In order to evaluate the health condition of the subject, the subject is usually asked to perform a fixed number of specific actions. Accordingly, the electronic device may determine whether the signal corresponding to the specific action in the analysis result is valid according to the fixed number, and may update the analysis result when the signal is determined to be invalid (such as the signal being caused by noise). In addition, the electronic device may also improve the analysis result according to the distance signal and the acceleration signal detected by the radar, so that the mapping relationship between the signal and the action contained in the analysis result is more accurate.

What is claimed is:

1. A method of processing a motion signal, comprising:
performing detection through a radar to obtain a dynamic signal;
executing continuous wavelet transform on the dynamic signal to obtain a scalogram;
dividing the scalogram to generate a plurality of samples;
clustering the samples into a first cluster and a second cluster;
sampling the motion signal from the dynamic signal according to the first cluster; and
outputting the motion signal.

2. The method according to claim 1, wherein sampling the motion signal from the dynamic signal according to the first cluster comprises:
determining a time window according to the first cluster; and
sampling the motion signal from the dynamic signal according to the time window.

3. The method according to claim 1, wherein executing the continuous wavelet transform on the dynamic signal to obtain the scalogram comprises:
executing singular spectrum analysis on the dynamic signal to reconstruct the dynamic signal; and
executing the continuous wavelet transform on the reconstructed dynamic signal to obtain the scalogram.

4. The method according to claim 1, further comprising:
calculating a slope of the motion signal to generate a slope signal;
determining that a first sampling signal in the slope signal corresponds to a first action to generate an analysis result; and
outputting the analysis result.

5. The method according to claim 4, wherein determining that the first sampling signal in the slope signal corresponds to the first action to generate the analysis result comprises:
in response to a time period of the first sampling signal being greater than a time threshold, determining that the first sampling signal is valid to generate the analysis result.

6. The method according to claim 4, wherein the slope signal further comprises a second sampling signal and a third sampling signal, wherein the first sampling signal and the third sampling signal correspond to a first classification, and the second sampling signal between the first sampling signal and the third sampling signal corresponds to a second classification, the method further comprising:
in response to a time period of the second sampling signal being less than or equal to a time threshold, updating the slope signal to match the first sampling signal, the second sampling signal, and the third sampling signal with the first action.

7. The method according to claim 6, further comprising:
in response to each sampling point of the first sampling signal being greater than a sampling threshold, determining that the first sampling signal corresponds to the first classification.

8. The method according to claim 6, further comprising:
in response to each sampling point of the second sampling signal being less than or equal to a sampling threshold, determining that the second sampling signal corresponds to the second classification.

9. The method according to claim 4, further comprising:
performing detection through the radar to obtain a distance signal;
normalizing the slope signal;
multiplying the normalized slope signal by the distance signal to generate a reference signal; and
updating the analysis result according to the reference signal.

10. The method according to claim 9, wherein updating the analysis result according to the reference signal comprises:
extracting a plurality of sampling signals from the reference signal, and calculating a plurality of similarities between the sampling signals;
classifying the sampling signals into a first classification and a second classification according to the similarities; and
determining that at least one sampling signal in the first classification is valid to update the analysis result.

11. The method according to claim 10, wherein calculating the similarities between the sampling signals comprises:
executing dynamic time warping on the sampling signals to obtain the similarities.

12. The method according to claim 10, wherein classifying the sampling signals into the first classification and the second classification according to the similarities comprises:
executing agglomerative hierarchical clustering on the sampling signals according to the similarities, so that a number of samples assigned to the first classification reaches a preset value.

13. The method according to claim 9, further comprising:
determining that a plurality of sampling signals in the slope signal respectively correspond to a plurality of actions; and
in response to a number of the actions being greater than a preset value, updating the analysis result according to the reference signal.

14. The method according to claim 4, further comprising:
performing detection through the radar to obtain a distance signal, wherein the distance signal comprises a second sampling signal corresponding to the first sampling signal; and
in response to the second sampling signal being less than or equal to a sampling threshold, determining that the first sampling signal is valid to generate the analysis result.

15. The method according to claim 4, further comprising:
performing detection through the radar to obtain an acceleration signal, wherein the acceleration signal comprises a second sampling signal corresponding to the first sampling signal; and
in response to the second sampling signal being greater than a sampling threshold, determining that the first sampling signal is valid to generate the analysis result.

16. The method according to claim 1, wherein a wavelet function of the continuous wavelet transform comprises a cgau6 function.

17. The method according to claim 1, further comprising:
clustering the samples according to k-means clustering.

18. The method according to claim 1, wherein the dynamic signal comprises one of a velocity signal, an acceleration signal, and a Doppler signal.

19. The method according to claim 1, further comprising:
configuring the radar, so that a value of the dynamic signal increases during execution of a first action.

20. An electronic device of processing a motion signal, comprising:
a transceiver, communicatively connected to a radar; and
a processor, coupled to the transceiver and configured to execute:
performing detection through the radar to obtain a dynamic signal;
executing continuous wavelet transform on the dynamic signal to obtain a scalogram;
dividing the scalogram to generate a plurality of samples;
clustering the samples into a first cluster and a second cluster;
sampling the motion signal from the dynamic signal according to the first cluster; and
outputting the motion signal through the transceiver.
